(19) United States
(12) Patent Application Publication
(10) Pub. No.: US 2026/0000865 A1
(43) Pub. Date: Jan. 1, 2026

(54) CATHETER ASSEMBLY

(71) Applicant: EDWARDS LIFESCIENCES CORPORATION, Irvine, CA (US)

(72) Inventor: Pablo Hernan Catania, Costa Mesa, CA (US)

(21) Appl. No.: 19/320,942

(22) Filed: Sep. 5, 2025

Related U.S. Application Data
(63) Continuation of application No. PCT/US2024/017106, filed on Feb. 23, 2024.
(60) Provisional application No. 63/488,514, filed on Mar. 5, 2023.

(51) Int. Cl. A61M 25/00; A61F 2/24; A61M 25/01

(52) U.S. Cl. CPC A61M 25/0054; A61F 2/2436; A61M 25/0136; A61M 25/0155

(57) ABSTRACT

A bendable catheter body having a plurality of links connected in series along a longitudinal axis. The plurality of links includes a first link having an annular first intermediate portion, a first male protrusion extending proximally from the intermediate portion, and a second male protrusion extending distally from the intermediate portion. The first male protrusion is configured to be received within a first female recess of a second link of the plurality of links and includes a first pair of engagement surfaces facing the intermediate portion and first end surface. The engagement surfaces are configured to engage a pair of second engagement surfaces of the second link to limit axial extension of the catheter shaft. The first end surface is configured to engage an edge surface of a second intermediate portion of the second link to limit axial compression of the catheter body.

### RELATED APPLICATIONS

The present application is a continuation of PCT patent application no. PCT/US2024/017106, filed on Feb. 23, 2024, which claims the benefit of U.S. Provisional Patent Application Ser. No. 63/488,514, filed on Mar. 5, 2023, titled “Catheter Assembly”, each of these applications being incorporated herein by reference in its entirety.

### BACKGROUND OF THE INVENTION

Endovascular delivery systems can be used in various procedures to deliver medical devices or instruments to a target location inside a patient's body that are not readily accessible by surgery or where access without surgery is desirable. The systems described herein can be used to deliver medical devices (stents, heart valve, grafts, clips, repair devices, valve treatment devices, etc.) to a location in a patient's body.

Access to a target location inside the patient's body can be achieved by inserting and guiding the delivery system through a pathway or lumen in the body, including, but not limited to, a blood vessel, an esophagus, a trachea, any portion of the gastrointestinal tract, a lymphatic vessel, to name a few. Catheters are known in the art and have been commonly used to reach target locations inside a patient's body.

In some procedures, a catheter is used to deliver a device for replacing, repairing and/or remodeling a native heart valve. The native heart valves (i.e., the aortic, pulmonary, tricuspid, and mitral valves) serve critical functions in assuring the forward flow of an adequate supply of blood through the cardiovascular system. These heart valves can be damaged, and thus rendered less effective, by congenital malformations, inflammatory processes, infectious conditions, or disease. Such damage to the valves can result in serious cardiovascular compromise or death. For many years the definitive treatment for such damaged valves was surgical repair or replacement of the valve during open heart surgery. However, open heart surgeries are highly invasive and are prone to many complications. Therefore, elderly and frail patients with defective heart valves often went untreated. More recently, transvascular techniques have been developed for introducing and implanting prosthetic devices in a manner that is much less invasive than open heart surgery. Transvascular techniques can be used for accessing the native mitral, aortic, tricuspid, and pulmonary valves.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The native tricuspid valve of the human heart connects the right atrium to the right ventricle. When operating properly, the leaflets of each heart valve function together as a one-way valve.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

Disclosed herein are delivery systems, steerable catheters, and related methods which can be used to deliver a medical device, tools, agents, or other therapy to a location within a body of a subject. The delivery system can include a catheter body having a plurality of links connected in series along a longitudinal axis and movable relative to each other. In some implementations, the first link is axially moveable relative to the second link. In some implementations, the first link is tiltable relative to the second link.

In some implementations the plurality of links includes a first link having an annular first intermediate portion, a first male protrusion extending proximally from the intermediate portion, and a second male protrusion extending distally from the intermediate portion. In some implementations, the first male protrusion includes a first pair of engagement surfaces facing the intermediate portion and a first end surface facing opposite the pair of engagement surfaces. In some implementations, first male protrusion is configured to be received within a first female recess of a second link of the plurality of links.

In some implementations, the pair of first engagement surfaces are configured to engage a pair of second engagement surfaces of the second link to limit axial extension of the catheter shaft. In some implementations, the first end surface is configured to engage an edge surface of a second intermediate portion of the second link to limit axial compression of the catheter body.

In some implementations, the first pair of engagement surfaces are perpendicular to the longitudinal axis. In some implementations, the first male protrusion has a head portion and a stem portion positioned between the head portion and the intermediate portion, wherein the head portion has a first width, and the stem portion has a second width less than the first width. In some implementations, the pair of first engagement surfaces connects the stem portion to the head portion.

In some implementations, the first male protrusion is one of a plurality of first male protrusions evenly spaced apart around a periphery of the intermediate portion. In some implementations, the plurality of first male protrusions includes four first male protrusions. In some implementations, the first male protrusion is T-shaped.

In some implementations, the second male protrusion is one of a plurality of second male protrusions evenly spaced apart around the periphery of the intermediate portion. In some implementations, the plurality of second male protrusions are circumferentially offset from the first male protrusions. In some implementations, the plurality of second male protrusions are circumferentially offset from the first male protrusions by 45 degrees.

In some implementations, the stem portion includes a first stem side surface and a second stem side surface opposite of and parallel to the first stem side surface. In some implementations, the head portion includes a first head side surface and a second head side surface opposite of and parallel to the first stem side surface.

In some implementations, the second link includes a third male protrusion extending distally from the second intermediate portion and a fourth male protrusion extending distally from the second intermediate portion. In some implementations the intermediate portion, the third male protrusion, and the fourth male protrusion form the first female recess of the second link. In some implementations, the pair of second engagement surfaces of the second link include one of the pair of second engagement surfaces on the third male protrusions and a second of the pair of second engagement surfaces on the fourth male protrusion. In some implementations, the pair of second engagement surfaces are parallel to each other.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of embodiments of the present disclosure, a more particular description of the certain embodiments will be made by reference to various aspects of the appended drawings. It is appreciated that these drawings depict only typical embodiments of the present disclosure and are therefore not to be considered limiting of the scope of the disclosure. Moreover, while the figures can be drawn to scale for some embodiments, the figures are not necessarily drawn to scale for all embodiments. Embodiments and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 is another cutaway view of the human heart in a systolic phase showing mitral regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIG. 8 is a perspective view of an example of a hypotube that provides structure and control of a flex section at a distal portion of a steerable catheter;

FIG. 9 is a schematic perspective cross-sectional view of the hypotube of FIG. 8;

FIG. 10 is a schematic front view of an example of a hypotube that forms a backbone of at least a part of a steerable catheter;

FIG. 11 is an end view of the hypotube of FIG. 10;

FIG. 12 is a schematic cross-sectional view of the hypotube of FIG. 10;

FIG. 13 illustrates 3-dimensional steering of an example of a catheter including the exemplary hypotube of FIGS. 10-11;

FIG. 14 is a front view of an example of a distal end of a steerable catheter;

FIG. 15 is a side view of the distal end of the steerable catheter of FIG. 14;

FIG. 16 is a cross-sectional view of the distal end of the steerable catheter of FIG. 14 taken along plane 29 of FIG. 15;

FIG. 17 is an enlarged view of the area 30 of FIG. 16;

FIG. 18 is a perspective view of the distal end of the steerable catheter of FIG. 14 with most of the springs and actuation elements removed;

FIG. 19 illustrates 3-dimensional steering of an example of a catheter including an exemplary steering mechanism;

FIG. 20 is a perspective view of an example of a distal end of a steerable catheter in a bent condition;

FIG. 21 is a side view of the distal end of the steerable catheter of FIG. 20;

FIG. 22 is a bottom view of the distal end of the steerable catheter of FIG. 20;

FIG. 23 is a front view of the distal end of the steerable catheter of FIG. 20;

FIG. 24 is a rear view of the distal end of the steerable catheter of FIG. 20;

FIG. 25 is a cross-sectional view of the distal end of the steerable catheter of FIG. 20 taken along plane B-B of FIG. 24;

FIG. 26 is a cross-sectional view of the distal end of the steerable catheter of FIG. 20 taken along plane C-C of FIG. 24;

FIG. 27 is a cross-sectional view of the distal end of the steerable catheter of FIG. 20 taken along plane A-A of FIG. 24;

FIG. 28 is a schematic view of the distal end of the steerable catheter of FIG. 20 shown in a bent condition during implantation of an implantable device;

FIG. 29 is a perspective view of an example catheter body in a linear configuration;

FIG. 30 is a perspective view of the catheter body of FIG. 29 in a bent configuration;

FIG. 31 is a perspective view of an example link for the catheter body of FIG. 29;

FIG. 32 is a side view of the link of FIG. 31;

FIG. 33 is an end view of the link of FIG. 31;

FIG. 34 is a plan view of a portion of a catheter body of FIG. 31 in a first stage of manufacture;

FIG. 35 is a plan view of the catheter body of FIG. 31 in a second stage of manufacture;

FIG. 36 is a plan view of the catheter body of FIG. 31 in a third stage of manufacture;

FIG. 37 is a partial plan view of the catheter body of FIG. 31 in an alternative first stage of manufacture;

FIG. 38 is a partial side view of the catheter body of FIG. 31 in a compressed condition; and

FIG. 39 is a is a partial side view of the catheter body of FIG. 31 in an extended condition.

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, various implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

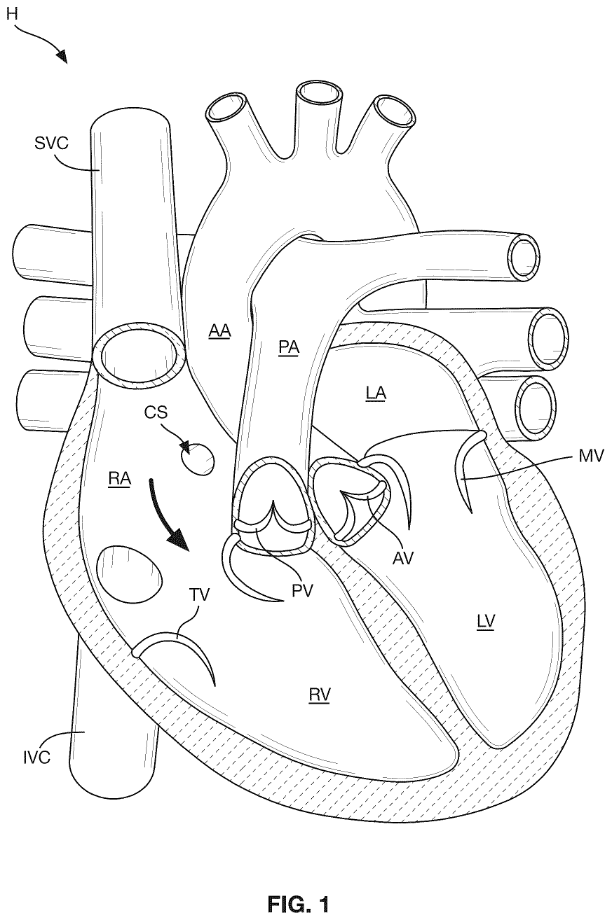

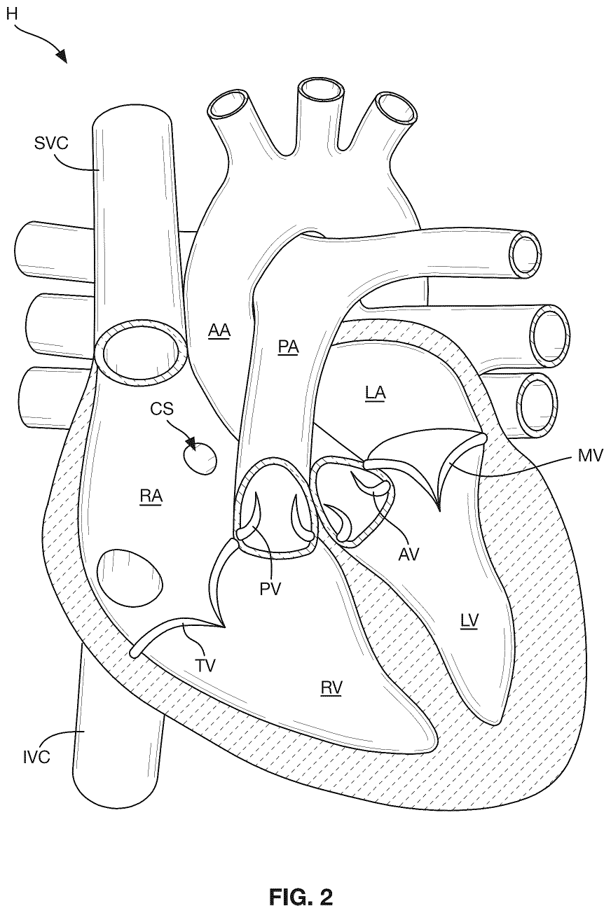

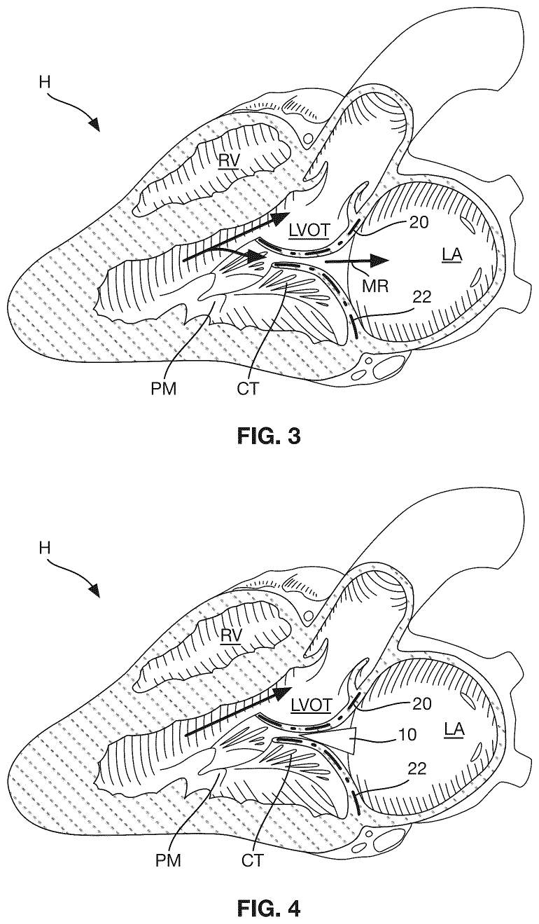

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV, respectively; i.e., the atrioventricular valves. The aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-7) extending inward across the respective orifices that come together or “coapt” in the flow stream to form the one-way, fluid-occluding surfaces. The devices and systems disclosed herein can be used to replace, repair, remodel, etc. the mitral valve MV, the tricuspid valve TV, the aortic valve AV, and/or the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV back into the left atrium LA and blood is collected in the left atrium from the pulmonary vein.

Referring now to FIGS. 1-7, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIG. 3, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV to prevent the mitral valve MV from being reverted. The mitral valve MV opens and closes in response to relative pressure changes in the left atrium LA and the left ventricle LV. The papillary muscles PM do not open or close the mitral valve MV. Rather, the papillary muscles PM support or brace the leaflets 20, 22 against the high pressure necessary to circulate blood throughout the body. Together the papillary muscles PM and the chordae tendineae CT are known as the subvalvular apparatus, which functions to keep the mitral valve MV from prolapsing into the left atrium LA when the mitral valve closes.

As seen from a Left Ventricular Outflow Tract (LVOT) view shown in FIG. 4, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus. As a result, the leaflets 20, 22 form a space having a generally triangular shape 10 that is annotated in FIG. 4.

Various disease processes can impair proper function of one or more of the native valves of the heart H. These disease processes include degenerative processes (e.g., Barlow's Disease, fibroelastic deficiency), inflammatory processes (e.g., Rheumatic Heart Disease), and infectious processes (e.g., endocarditis). In addition, damage to the left ventricle LV or the right ventricle RV from prior heart attacks (i.e., myocardial infarction secondary to coronary artery disease) or other heart diseases (e.g., cardiomyopathy) can distort a native valve's geometry, which can cause the native valve to dysfunction.

Generally, a native valve may malfunction in two different ways: (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. The second type of valve malfunction, valve regurgitation, occurs when the leaflets of the valve do not close completely thereby causing blood to leak back into the prior chamber (e.g., causing blood to leak from the left ventricle to the left atrium).

There are three mechanisms by which a native valve becomes regurgitant—or incompetent-which include Carpentier's type I, type II, and type III malfunctions. A Carpentier type I malfunction involves the dilation of the annulus such that normally functioning leaflets are distracted from each other and fail to form a tight seal—i.e., the leaflets do not coapt properly. Included in a type I mechanism malfunction are perforations of the leaflets, as are present in endocarditis. A Carpentier's type II malfunction involves prolapse of one or more leaflets of a native valve above a plane of coaption. A Carpentier's type III malfunction involves restriction of the motion of one or more leaflets of a native valve such that the leaflets are abnormally constrained below the plane of the annulus. Leaflet restriction can be caused by rheumatic disease (Ma) or dilation of a ventricle (IIIb).

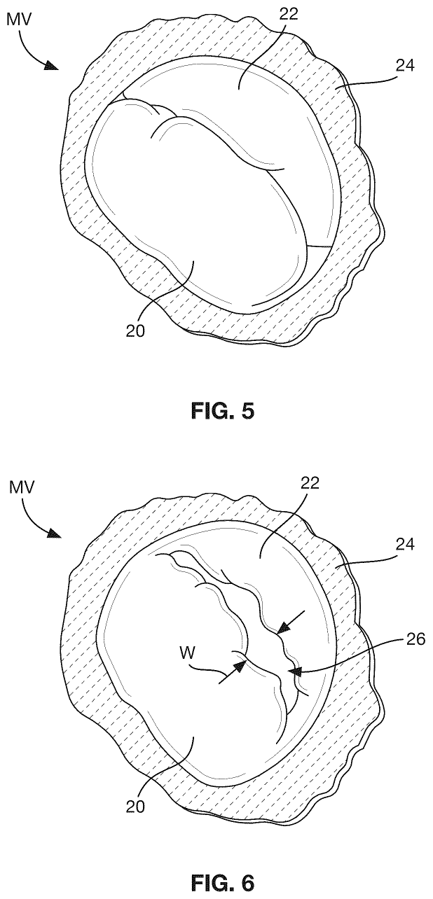

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22 that allows blood to flow back into the left atrium LA from the left ventricle LV during systole, as illustrated by the mitral regurgitation MR flow path shown in FIG. 6. As set forth above, there are several different ways that a leaflet (e.g. leaflets 20, 22 of mitral valve MV) may malfunction such that mitral regurgitation MR occurs.

Although stenosis or regurgitation can affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart is primarily responsible for circulating the flow of blood throughout the body, substantially higher pressures are experienced by the left side heart structures (i.e., the left atrium LA, the left ventricle LV, the mitral valve MV, and the aortic valve AV). Accordingly, malfunction of the mitral valve MV or the aortic valve AV is particularly problematic and often life threatening.

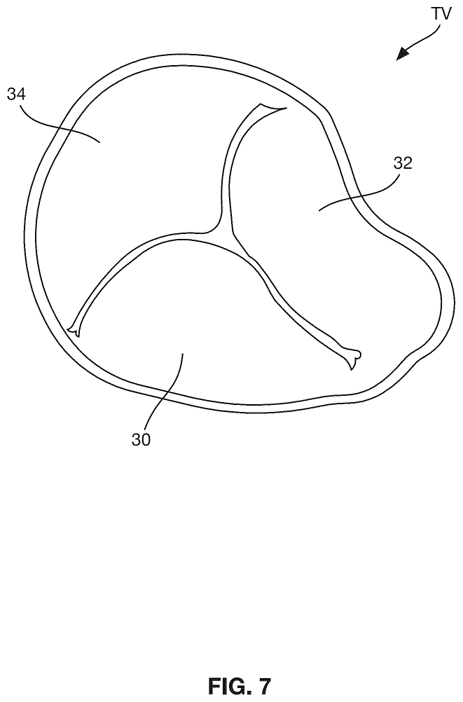

Malfunctioning native heart valves may either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, the most conventional treatments for a stenotic aortic valve or stenotic pulmonary valve are removal and replacement of the valve with a surgically implanted heart valve, or displacement of the valve with a transcatheter heart valve. The mitral valve MV and the tricuspid valve TV (FIG. 7) are more prone to deformation of annulus and/or leaflets, which, as described above, prevents the mitral valve MV or tricuspid valve TV from closing properly and allows for regurgitation or back flow of blood from the ventricle into the atrium (e.g., a deformed mitral valve MV may allow for mitral regurgitation MR or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation can occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV.

The devices and concepts provided herein can be used to replace any native valve, repair any native valve, remodel any native valve, as well as any component of a native valve. In one non-limiting example, a valve repair device can be used on native mitral leaflets 20, 22 or tricuspid leaflets 30, 32, 34.

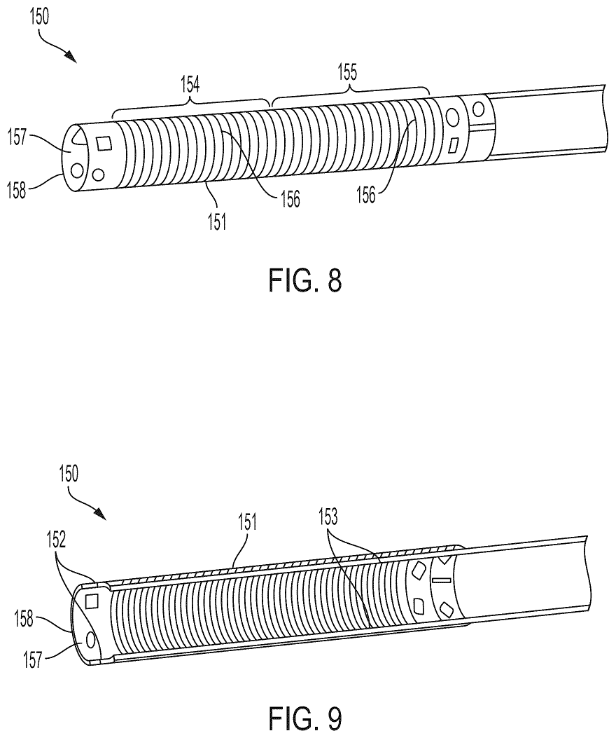

Referring now to FIGS. 8-9, an exemplary steerable catheter 150 is shown that includes a hypotube 151 arranged at a distal portion of the catheter 150 to provide structure and control of the flex section at the distal portion of the catheter 150. The catheter 150 can include at least one shaft that includes the hypotube 151 and relies on the hypotube 151 as a backbone or spine of the entire shaft. The hypotube 151 can be co-centric with the rest of the catheter shaft. And can extend for the entire length or substantially the entire length of the catheter shaft. Optionally, the hypotube 151 can be located only at a distal portion or distal end of the shaft. A distal end 158 of the hypotube 151 can be aligned with or spaced apart from the distal portion or end of the catheter 150.

The hypotubes described herein, such as, for example, the hypotube 151, can be constructed using any suitable metal or alloy, such as, for example, stainless steel, nitinol, titanium, and the like. The hypotube 151 can also include an internal liner made from a material having substantially same properties as the material used to make lumens used in conjunction with other structures of the catheter, such as actuation elements (e.g., pull wires) or compression members (e.g., compression coils). Additionally, a reflowed jacket or outer material can be provided over the entirety of the catheter shaft.

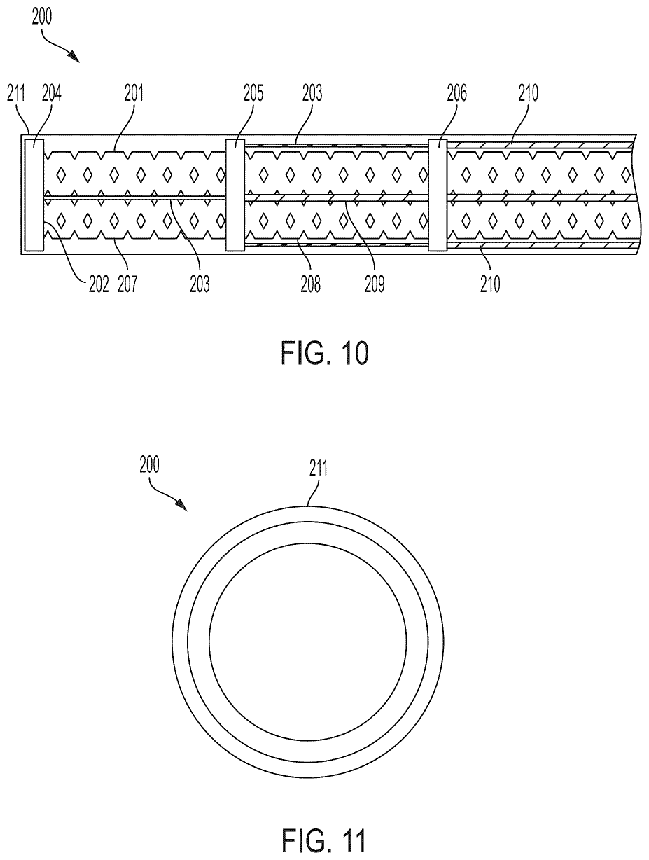

An operator of the catheter 150 can bend the distal portion of the catheter 150 by pulling or releasing actuation elements 153 that extend along the length of the hypotube 151. The actuation elements 153 can be pull wires having a circular or flat rectangular cross-sectional shape and can be secured to the hypotube 151 via a pull ring (see, e.g., FIG. 10) or via attachment locations 152. The actuation elements 153 can be directly attached to the attachment locations via welding, an adhesive, a mechanical fastener, or the like. For example, the actuation elements 153 can be laser welded to the attachment locations 152 of the hypotube 151.

A first or anchor portion 157 of the hypotube 151 proximal to the distal end 158 of the hypotube 151 can serve the same function as a pull-ring, that is, to attach to the actuation elements 153 so that tension applied to the actuation elements 153 is transmitted to the hypotube 151. Integrating a pull ring structure into the hypotube 151 prohibits misalignment of the pull ring and hypotube 151 that can occur when two separate components are joined together. The anchor portion 157 can be formed from an area of the hypotube 151 with a particular shape and/or increased strength, rigidity, and thickness to provide sufficient strength for receiving the actuation elements 153. To provide added stiffness and strength, the wall thickness of the anchor portion 157 of the hypotube 151 proximal to the distal end 158 of the hypotube 151 can be greater than the wall thickness of the remainder of the hypotube 151. For example, a thickness of a wall of an anchor portion 157 of the hypotube can be in the range of about 0.5 mm to about 2.5 mm. The anchor portion 157 of the hypotube 151 can also be wider than the remainder of the hypotube 151, the anchor portion 157 having a diameter in a range of about 5 mm to about 10 mm.

The hypotube 151 can be formed from a single section that bends substantially uniformly when actuated by the actuation elements 153. To facilitate bending, the hypotube 151 can include a plurality of relief cuts 156 that allow the hypotube 151 to flex and bend in one or more flexing directions with little or no axial compression under load. The relief cuts 156 can be formed in the hypotube 151 via laser cutting or any other suitable cuttings means to alter the bending characteristics of the hypotube 151. The relief cuts 156 can be formed in a variety of patterns, e.g., straight, spiral, staggered, zigzag, etc. In one example, the repeating cuts 156 are aligned in a straight line along the axis of the shaft of the catheter. In one example, the repeating cuts 156 are staggered along the axis of the shaft of the catheter.

The hypotube 151 can include sections having different bending characteristics: that is, a first section 154 arranged near the distal end 158 of the hypotube 151 and a second section 155 proximal of the first section. That is, the first and second sections 154, 155 can differ in bend direction, bend rate, and bend radius when tension is applied to the actuation elements 153 to actuate the hypotube 151. The different bending characteristics of the first and second sections 154, 155 of the hypotube 151 can be provided in a wide variety of ways, such as, for example, by varying the thickness, stiffness, and material type of the first and second sections 154, 155.

The structure of the first and second sections 154, 155 can also be varied via the relief cuts 156 along the hypotube 151. The relief cuts 156 can be changed in their size, shape, and spacing along the length of the hypotube 151 and, in particular, between the first and second sections 154, 155 to provide different bending characteristics between the first and second sections 154, 155. For example, the bending direction can be altered between the first and second sections 154, 155. The spacing between consecutive cuts in the first section 154 can be greater than, the same as, or lesser than the spacing between consecutive cuts in the second section 155. In some examples, the relief cuts 156 are formed such that links or link-like formations are formed in the hypotube 151.

Referring now to FIGS. 10-13, an exemplary delivery system 200 is shown that includes a catheter 211 and a hypotube 201 arranged within the catheter 211. The hypotube 201 extends along the catheter 211 to a distal end 202 and can be caused to bend or flex via the actuation of a plurality of actuation elements 203 arranged around the circumference of the catheter 211. The hypotube 201 can be divided longitudinally into sections that can be independently actuated by the actuation elements 203. In some examples, the bending characteristics from one section of the hypotube 201 to another can be different.

The hypotube 201 includes first, second, and third ring sections 204, 205, 206 that provide support to the actuation elements 203 and also attachment locations for attaching the actuation elements 203 to the hypotube 201. The first ring section 204 is arranged at the distal end 202 of a first section 207 of the hypotube 201, the second ring section 205 is arranged at a mid-section between the first section 207 and a second section 208, and the third ring section 206 is arranged at a proximal end of the second section 208. While three ring sections 204, 205, 206 are shown in FIG. 2, the hypotube can include any suitable number of ring sections, such as, for example, 2, 3, 4, 5, or more ring sections that can correspond to a similar number of articulable sections of the hypotube 201.

The hypotube 201 can be formed from a tube of material or a sheet of material that is rolled and welded or otherwise joined along a seam. The tube or sheet of material can have a plurality of spaced apart cutouts arranged in a grid and each having a diamond shape (see FIG. 10) or any other suitable shape. For example, the cutouts can be formed as slits that extend around a majority of the circumference of the hypotube 201 to form a series of links having a rib cage like configuration. In some examples, the links include a slot formed between each pair of adjacent links, a bottom orifice for each link, and at least one slit extending upward from the bottom orifice.

The ring sections 204, 205, 206 can have an outer diameter that is the same as the outer diameter of the hypotube 201. The outer diameter of one or more of the ring sections 204, 205, 206 can also be larger than the outer diameter of the hypotube 201 and smaller than an inside diameter of the catheter shaft—i.e., the diameter of the ring sections 204, 205, 206 can be larger than the diameter of the hypotube 201 yet be small enough to fit within the catheter 211. The ring sections 204, 205, 206 can optionally be integrally formed in the hypotube 201 by cutting or otherwise forming the ring sections 204, 205, 206 in the material of the hypotube 201. Optionally, the ring sections 204, 205, 206 can be separate rings that are attached to the outer surface of the hypotube 201 via any suitable attachment means, such as, for example, welding, an adhesive, mechanical fastening, or the like.

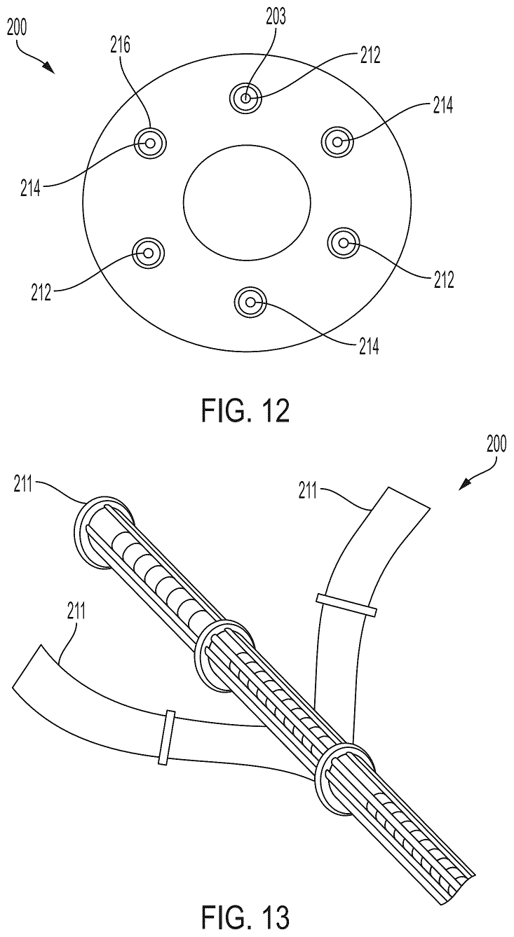

The actuation elements 203 are arranged into two groups: a first group 212 (FIG. 12) for articulating the first section 207 and a second group 214 for articulating the second section 208. The actuation elements 203 of the first group 212 extend through openings 216 in the second and third ring sections 205, 206 to attach to the first pull ring 204. The actuation elements 203 of the first group 212 extend through compression members 209 that terminate at the second pull ring 205. The actuation elements 203 of the second group 214 extend through openings 216 in the third ring section 206 to attach to the second pull ring 205. The actuation elements 203 of the second group 214 extend through compression members 210 that terminate at the third pull ring 206.

As can be seen in FIG. 12, the actuation elements 203 in each of the first and second groups 212, 214 are radially spaced apart from each other by about 120 degrees so that the three actuation elements 203 in each group are evenly spaced around the circumference of the hypotube 201. Optionally, one of the three actuation elements 203 in the first or second groups 212, 214 can be spaced apart about 135 degrees from the other two actuation elements 203. Additional actuation elements 203 can also be included such that the actuation elements 203 are radially spaced apart from other actuation elements 203 by about 90 degrees, or about 60 degrees, or about 30 degrees.

Applying tension to the actuation elements 203 causes the attached ring section 204, 205, 206 to move or tilt in the direction of the net tension force applied to the ring section 204, 205, 206. Consequently, the first or second section 207, 208 of the hypotube 201 that is immediately proximal of the articulated ring section 204, 205 is caused to flex or bend toward the applied force. Bending forces applied to one side of the hypotube 201 via one of the three actuation elements 203 in one of the first and second groups 212, 214 can be counteracted by forces applied to the other two actuation elements 203 in the first or second group 212, 214.

Providing three actuation elements 203 in each of the first and second groups 212, 214 enables each of the first and second sections 207, 208 to be articulated in any direction around the hypotube 201 by varying the amount of tension applied to each of the actuation elements 203. In particular, the direction of the bend in the hypotube 201 depends on the proportional distribution of tension forces in each of the three actuation elements 203 of the first or second groups 212, 214. That is, the relative proportion of tension applied to each of the three actuation elements 203—independent of the amount of tension applied—determines bend direction. The amount of tension applied to the actuation elements 203, however, is directly related to the magnitude of the bend in the hypotube 201; the greater the tension imbalance the greater the bend magnitude (i.e., the tighter or smaller the bend radius). Consequently, the catheter 211 of the delivery system 200 can be articulated into a wide variety of positions, such as, for example, the range of positions shown in FIG. 13.

The actuation elements from each group 212, 214 can be connected to one or more steering elements of a steering mechanism, such as example steering mechanisms disclosed herein, that is arranged in a handle (not shown) at a proximal end of the catheter. For example, a first steering mechanism can be used to control the bending of the first section 207 and a second steering mechanism can be used to control the bending of the second section 208. Optionally, a single steering mechanism can control both of the first and second sections 207, 208. The steering mechanisms can be arranged in a single handle or in multiple handles such that each handle contains a single steering mechanism.

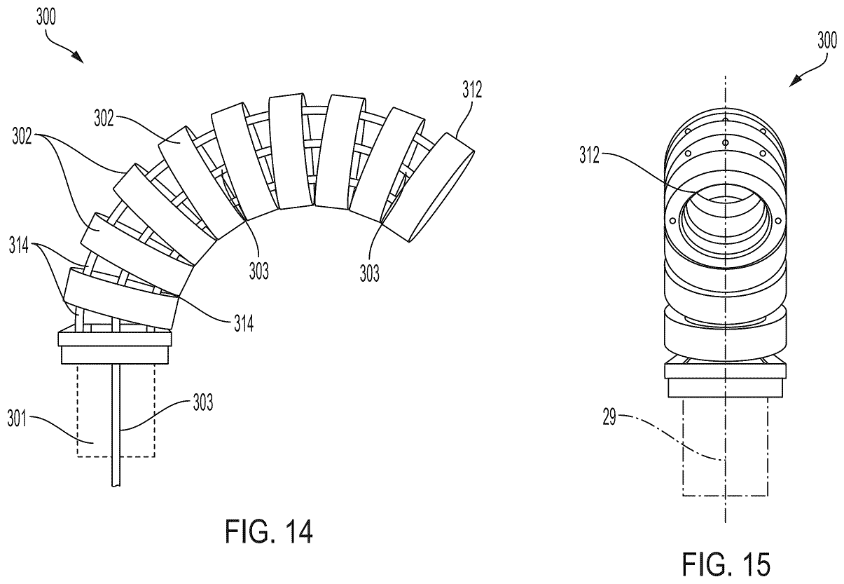

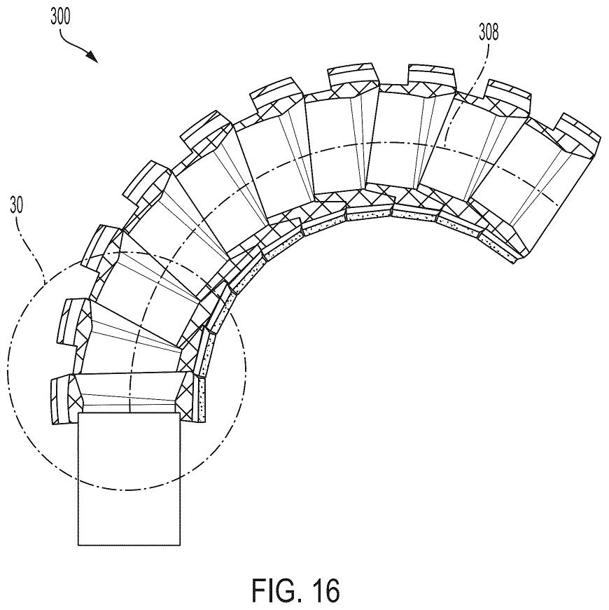

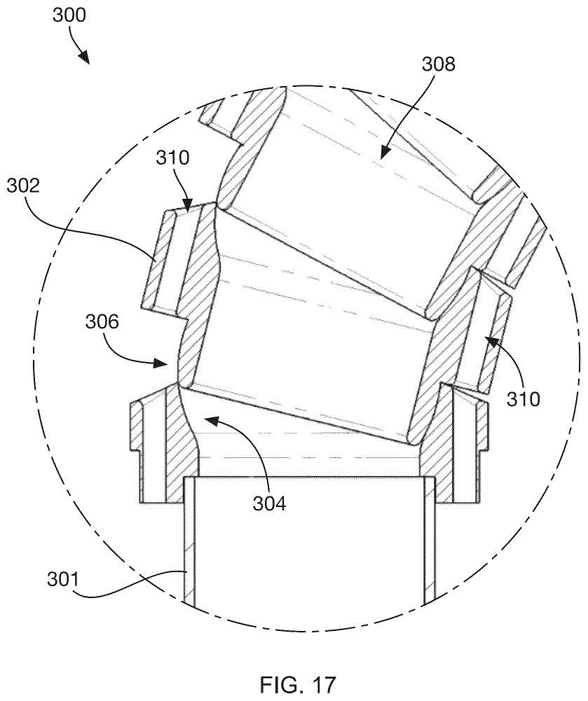

Referring now to FIGS. 14-18, an exemplary delivery system 300 is shown. The delivery system 300 is formed from a plurality of links 302 arranged at a distal end of a catheter shaft 301. The links 302 operate similar to vertebrae of the human spine in that the links 302 include male and female connecting surfaces 304, 306 that provide a sliding joint between adjacent links 302. The connecting surfaces 304, 306 can be formed in a ball and socket joint configuration that allows the links 302 to pivot relative to one another. Each link 302 includes a central opening or lumen 308 and openings or lumen 310 for guiding and supporting actuation elements 303 that extend along the length of the delivery device 300 to a distal end 312. An optional hypotube (not shown) can also be provided that extends through the central openings 308 of the links 302.

The actuation elements 303 and actuation element openings 310 are radially spaced apart from each other by about 90 degrees to accommodate four actuation elements 303 extending along the length of the device 300 to the distal end 312. Applying tension to the actuation elements 303 causes the distal end 312 and the links 302 to move or tilt in the direction of the net tension force applied to the actuation elements 303. Consequently, the plurality of links 302 are caused to pivot relative to each other so that the device 300 flexes or bends toward the applied force. Bending forces applied to one side of the device 300 via one of the three actuation elements 303 can be counteracted by forces applied to the other actuation elements 303.

Providing four actuation elements 303 around the circumference of the device 300 enables the device 300 to be articulated in any direction around by varying the amount of tension applied to each of the actuation elements 303. In particular, the direction of the bend in the device 300 depends on the proportional distribution of tension forces in each of the four actuation elements 303. That is, the relative proportion of tension applied to each of the four actuation elements 303—independent of the amount of tension applied—determines bend direction. The amount of tension applied to the actuation elements 303, however, is directly related to the magnitude of the bend in the device 300; the greater the tension imbalance the greater the bend magnitude (i.e., the tighter or smaller the bend radius). Consequently, the end of the catheter shaft 301 of the delivery system 300 can be articulated into a wide variety of positions.

The actuation elements 303 can be connected to one or more steering elements of a steering mechanism, such as an exemplary steering mechanisms disclosed herein, that is arranged in a handle (not shown) at a proximal end of the catheter. For example, a first steering mechanism can be used to control the bending in a first bending plane of the device 300 so that the steering mechanism is connected to two actuation elements 303 that are spaced apart by 180 degrees around the device 300 and a second steering mechanism can be used to control the bending of the device 300 in a second bending plane that is orthogonal to the first bending plane. Optionally, a single steering mechanism can control bending in both the first and second bending planes. The steering mechanisms can be arranged in a single handle or in multiple handles such that each handle contains a single steering mechanism.

The actuation elements 303 can extend through compression members or coils (not shown) that extend from a handle or proximate the handle to a proximal portion of the most proximal link 302. The proximal side of the link 302 can include pockets or recesses for receiving a distal end of the compression member. Optionally, the compression member can run the entire length of the catheter shaft 301. In any of the catheter implementations herein, each compression member can run through an individual lumen in a shaft of the catheter so that flexing of the shaft does not hinder independent movement of the compression member. In one implementation, the proximal face of a hypotube and/or links of a hypotube have bores and/or extensions to accept or abut against the compression members. The proximal face of the most proximal link 302 can also have bores and/or extensions to accept or abut against the compression members.

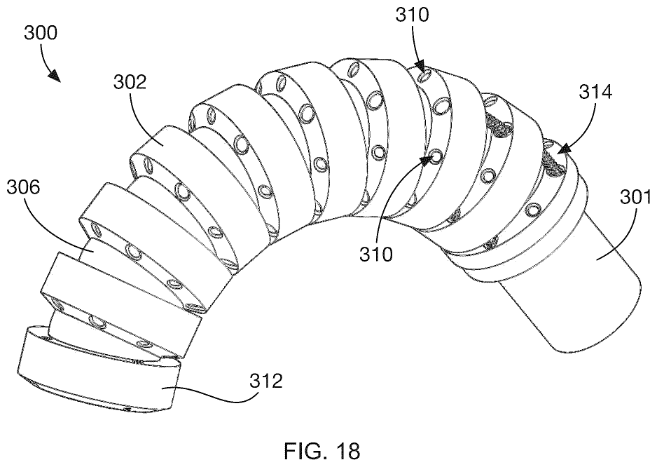

The device 300 can further include stiffening members arranged between the links 302. The stiffening members cause the device 300 to be biased in an extension direction so that the links 302 tend to straighten out after tension applied to the actuation elements 303 is relieved. The stiffening members can be formed in a tube shape from a shape-memory alloy, such as nitinol. As can be seen in FIG. 18, the stiffening members can be springs 314 that are biased in an expanding direction so that as the device 300 tends to straighten as tension applied to the actuation elements 303 is relieved. The springs 314 can be arranged between each pair of adjacent links 302 or can extend through multiple links 302. Four springs 314 can be arranged between each pair of adjacent links 302 so that the springs 314 are radially spaced apart by about 90 degrees and can be arranged between adjacent actuation elements 303 so that the springs 314 and actuation elements 303 alternate around the circumference of the device 300. Evenly spacing the springs 314 around the circumference of the links 302 evens out the forces applied to the links 302 and helps to maintain a symmetrical distance between adjacent links 302.

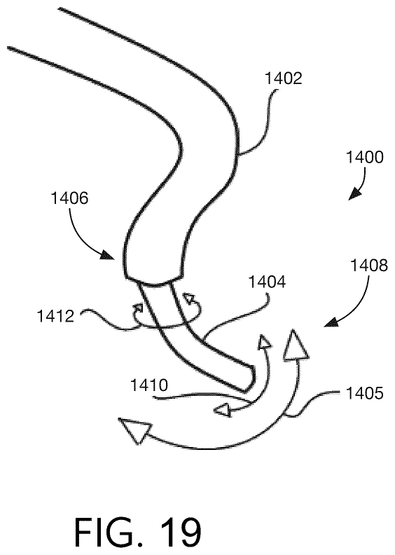

Referring now to FIG. 19, an example steerable catheter 1400 is shown that includes two catheter shafts: an outer shaft 1402 and an inner shaft 1404. The outer shaft 1402 is configured to reach a location above a center of an annulus, e.g., a mitral valve and a tricuspid valve annulus. The outer shaft 1402 has at least two sequentially arranged bending sections (see, e.g., FIGS. 8-13) so that the outer shaft 1402 can be bent and flexed so that a distal end 1406 of the outer shaft 1402 is arranged above and facing the native valve of a patient during an operation. Once the position of the outer shaft 1402 has been fine-tuned, the inner shaft 1404 can be extended, bent, and rotated to place a distal end 1408 of the inner shaft 1404 at a desired location closer to the tissue of the native valve.

The inner shaft 1404 can be controlled to extend distally so that the inner shaft 1404 becomes longer than the outer shaft 1402. Once extended to a desired length, the inner shaft 1404 can be manipulated by a steering mechanism to bend in a lateral direction 1410 and to twist or rotate in an axial direction 1412. These motions can be combined to move the extended and bent inner shaft 1404 in a sweeping motion 1405. Thus, rather than manipulating the distal end 1408 of the inner shaft 1404 via bending in two planes (similar to a cartesian coordinate system) the distal end 1408 of the inner shaft 1404 can be manipulated by bending and rotating (similar to a polar or spherical coordinate system). That is, the position of the distal end 1408 of the inner shaft 1404 can be specified by the extension length, the bend angle, and the rotation or twist angle of the inner shaft 1404. Thus, accessing a different location along the annulus of the native heart valve is a matter of rotating or twisting the inner shaft 1404 in the axial rotation direction 1412 a desired amount while maintaining the same bend angle and extension distance.

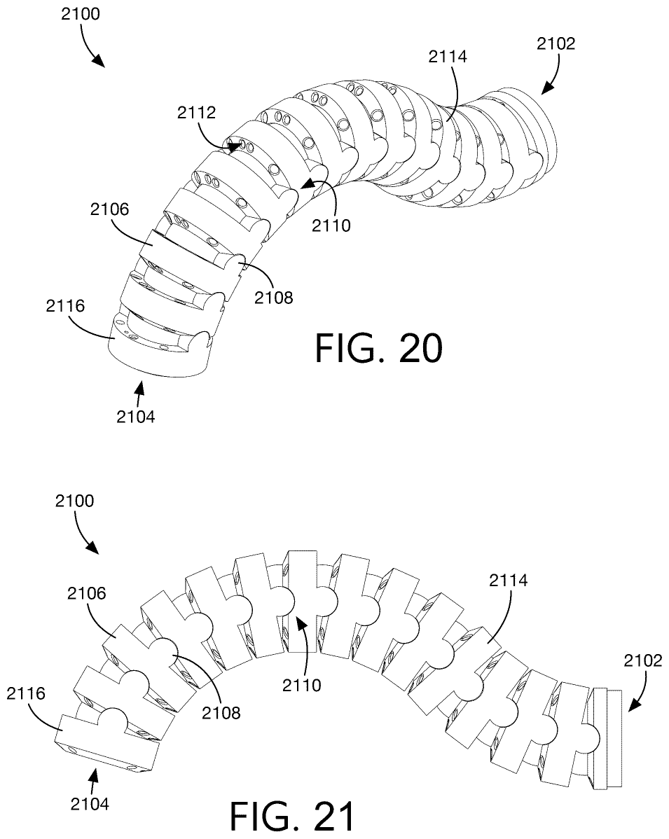

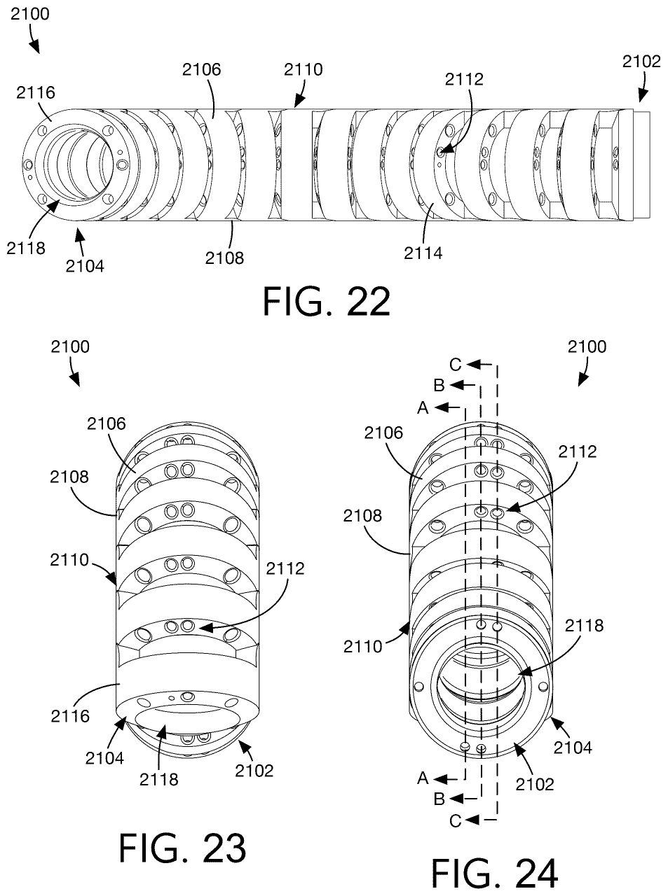

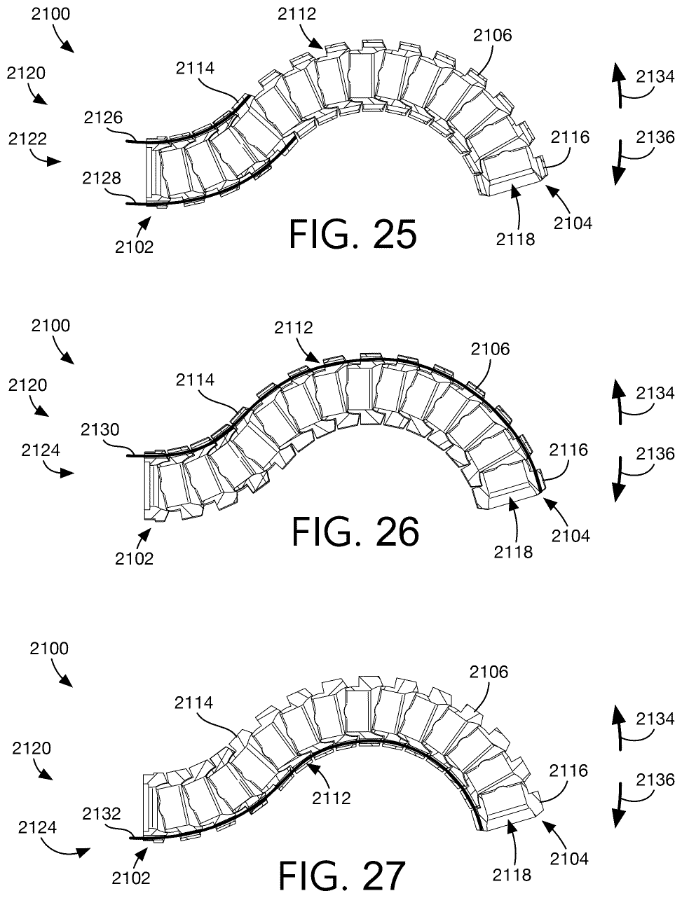

Referring now to FIGS. 20-28, an exemplary delivery system 2100 is shown. The delivery system 2100 extends from a proximal end 2102 to a distal end 2104 and is formed from a plurality of links 2106 arranged at a distal end of a catheter shaft (not shown). The links 2106 operate similar to vertebrae of the human spine in that the links 2106 include convex male protrusions 2108 that fit together with concave female recesses 2110 to provide a pivoting joint between adjacent links 2106. Each link 2106 includes a central opening or lumen 2118 and openings or lumen 2112 for guiding and supporting actuation elements 2120 (FIGS. 25-27) that articulate the delivery device 2100. An optional hypotube (not shown) can also be provided that extends through the central openings 2118 of the links 2106.

The convex male protrusions 2108 and concave female recesses 2110 prohibit relative rotation of the links 2106 except along the longitudinal axis of the delivery system 2100 (i.e., along the line B-B of FIG. 24). The actuation elements 2120 can be actuated to bend a portion or all of the delivery system 2100 in a first direction 2134 and/or a second direction 2136. Applying tension to the actuation elements 2120 causes the link 2106 to which the actuation elements 2120 are attached to move or tilt in the direction of the net tension force applied to the actuation elements 2120. Consequently, the plurality of links 2106 are caused to pivot relative to each other so that the device 2100 flexes or bends toward the applied force. In other words, the relative proportion of tension applied to each of two opposingly arranged actuation elements 2120—independent of the amount of tension applied—determines bend direction. The amount of tension applied to the actuation elements 2120, however, is directly related to the magnitude of the bend in the device 2100; the greater the tension imbalance the greater the bend magnitude (i.e., the tighter or smaller the bend radius). The length of the delivery system 2100 bent by the actuation elements 2120 depends on the link 2106 to which the actuation elements 2120 are connected. That is, applying tension to one of the actuation elements 2120 applies bending forces to the links 2106 arranged proximate to the connected link 2106. Thus, the end of the catheter of the delivery system 2100 can be articulated into a wide variety of positions.

Referring now to FIG. 25, a first pair of actuation elements 2122 extends through the proximal end 2102 of the delivery system 2100 to a transition link 2114. The first pair of actuation elements 2122 includes a first actuation element 2126 and a second actuation element 2128 that are connected to opposite sides of the transition link 2114. The first actuation element 2126 and the second actuation element 2128 extend through lumen 2112 arranged along a central plane of the delivery system 2100, as can be seen in FIG. 24. Tension applied to the first actuation element 2126 and the second actuation element 2128 actuates or bends the delivery system 2100 between the proximal end 2102 and the transition link 2114 in the direction of the greater of the two tension forces applied to the first actuation element 2126 and the second actuation element 2128. That is, greater tension applied to the first actuation element 2126 bends the transition link 2114 in the first direction 2134 and greater tension applied to the second actuation element 2128 bends the transition link in the second direction 2136.

Referring now to FIGS. 26 and 27, a second pair of actuation elements 2124 extends through the proximal end 2102 of the delivery system 2100 to a distal link 2116 at the distal end 2104 of the delivery system 2100. The second pair of actuation elements 2124 includes a third actuation element 2130 and a fourth actuation element 2132 that are connected to opposite sides of the distal link 2116. The third actuation element 2130 and the fourth actuation element 2132 extend through lumen 2112 arranged adjacent to a central plane of the delivery system 2100, as can be seen in FIG. 24. Tension applied to the third actuation element 2130 and the fourth actuation element 2132 actuates or bends the delivery system 2100 between the proximal end 2102 and the distal link 2116 in the direction of the greater of the two tension forces applied to the third actuation element 2130 and the fourth actuation element 2132. That is, greater tension applied to the third actuation element 2130 bends the distal link 2116 in the first direction 2134 and greater tension applied to the fourth actuation element 2132 bends the transition link in the second direction 2136.

The second pair of actuation elements 2124 can be actuated to bend the entire length of the delivery system 2100 to move the distal end 2116 in the first direction 2134 or the second direction 2136. When only the second pair of actuation elements 2124 is actuated to bend the delivery system 2100, the distal end 2104 is displaced laterally away from the plane of the delivery system 2100 extending from the proximal end 2102. As can be seen in FIGS. 25-27, actuating one of the actuation elements 2120 of the first pair of actuation elements 2122 (i.e., the first actuation element 2126) in an opposite direction from the actuation element 2120 of the second pair of actuation elements 2124 (i.e., the fourth actuation element 2132) maintains the distal end 2104 of the delivery system 2100 in nearly the same plane as the proximal end 2102 of the delivery system 2100.

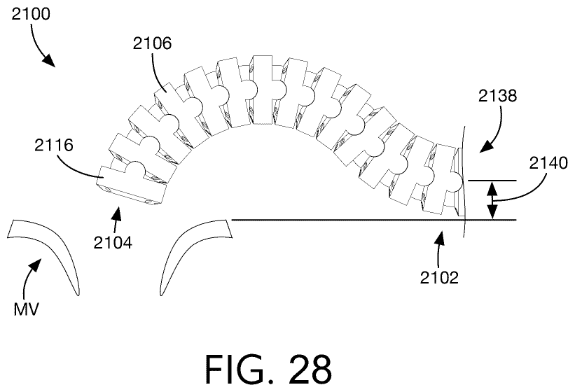

Referring now to FIG. 28, a schematic side view of the delivery system 2100 is shown protruding through a septal puncture or opening 2138 in the septum of the heart and bending toward the mitral valve MV. The trans-septal delivery technique is one technique that can be used to deliver implantable prosthetic devices within the mitral valve. During the trans-septal technique, the delivery device 2100 is extended through the inferior vena cava IVC (see FIGS. 1 and 2) and then through the septal puncture or opening 2138. The height of the distal end of the device 2100 when bent to a maximum bending condition (e.g., 90 degrees) determines a minimum distance between the mitral valve and the puncture through the septum that is made during implantation, i.e., a septal puncture height 2140. If the puncture through the septum is made too close to the mitral valve-below the minimum septal puncture height—the distal end will not be able to bend to 90 degrees without contacting the tissue of the heart, thereby frustrating proper alignment and implantation of the implantable prosthetic device in the mitral valve. The ability to bend the delivery system 2100 so that the distal end 2104 is in or is nearly in the same plane as the proximal end 2102 enables delivery of an implantable device during the trans-septal delivery technique when the septal puncture or opening 2138 is made at or below the minimum desired septal puncture height 2140.

The actuation elements 2120 can be connected to one or more steering elements of a steering mechanism, such as any of the exemplary steering mechanisms disclosed herein, that is arranged in a handle (not shown) at a proximal end of the catheter. For example, a first steering mechanism can be used to control the bending of the device 2100 between the proximal end 2102 and the transition link 2114 and a second steering mechanism can be used to control the bending of the device 2100 between the transition link 2114 and the distal link 2116. In this scenario, the first steering mechanism is connected to the first actuation element 2126 and the second actuation element 2128 that are spaced apart by 180 degrees around the device 2100 and the second steering mechanism is connected to the third actuation element 2130 and the fourth actuation element 2132 that are also spaced apart by 180 degrees around the device 2100. While two different steering mechanisms are used in this arrangement, both steering mechanisms control bending of the device 2100 in the same plane because of the convex male protrusions 2108 and concave female recesses 2110, as described above. Optionally, a single steering mechanism can control bending of the device up to the transition link 2114 and between the transition link 2114 and the distal link 2116. The steering mechanism or mechanisms can be arranged in a single handle or in multiple handles such that each handle contains a single steering mechanism.

The actuation elements 2120 can extend through compression members or coils (not shown) that extend from a handle or proximate the handle to a proximal portion of the most proximal link 2106. The proximal side of the link 2106 can include pockets or recesses for receiving a distal end of the compression member. Optionally, the compression member can run the entire length of the catheter shaft. In any of the catheter implementations herein, each compression member can run through an individual lumen in a shaft of the catheter so that flexing of the shaft does not hinder independent movement of the compression member. In one implementation, the proximal face of a hypotube and/or links of a hypotube have bores and/or extensions to accept or abut against the compression members. The proximal face of the most proximal link 2106 can also have bores and/or extensions to accept or abut against the compression members.

The device 2100 can further include stiffening members (not shown) arranged between the links 2106. The stiffening members cause the device 2100 to be biased in an extension direction so that the links 2106 tend to straighten out after tension applied to the actuation elements 2120 is relieved. The stiffening members can be formed in a tube shape from a shape-memory alloy, such as nitinol. Like the springs 314 of the device 300 shown in FIG. 18, the stiffening members can be springs that are biased in an expanding direction so that as the device 2100 tends to straighten as tension applied to the actuation elements 2120 is relieved. The springs can be arranged between each pair of adjacent links 2106 or can extend through multiple links 2106. Four springs can be arranged between each pair of adjacent links 2106 so that the springs are radially spaced apart by about 90 degrees. Evenly spacing the springs around the circumference of the links 2106 evens out the forces applied to the links 2106 and helps to maintain a symmetrical distance between adjacent links 2106.

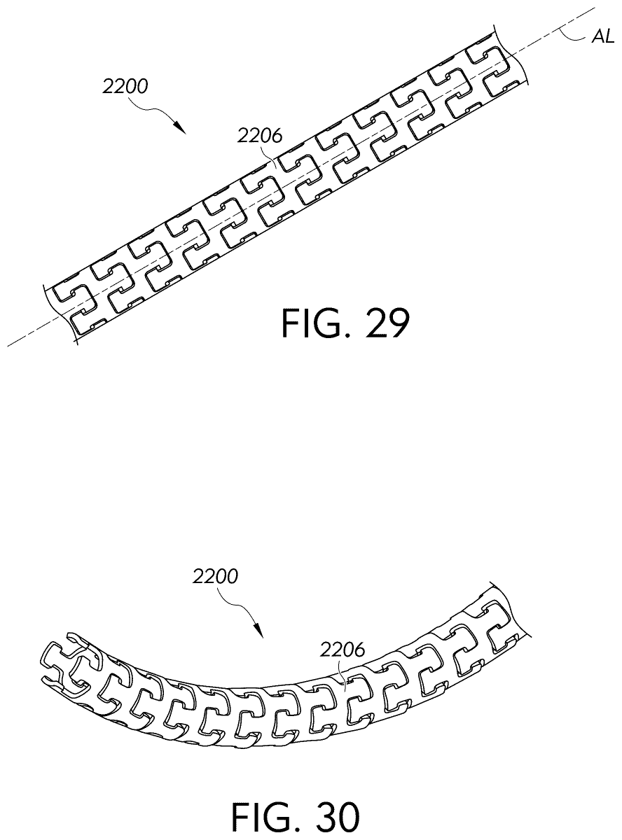

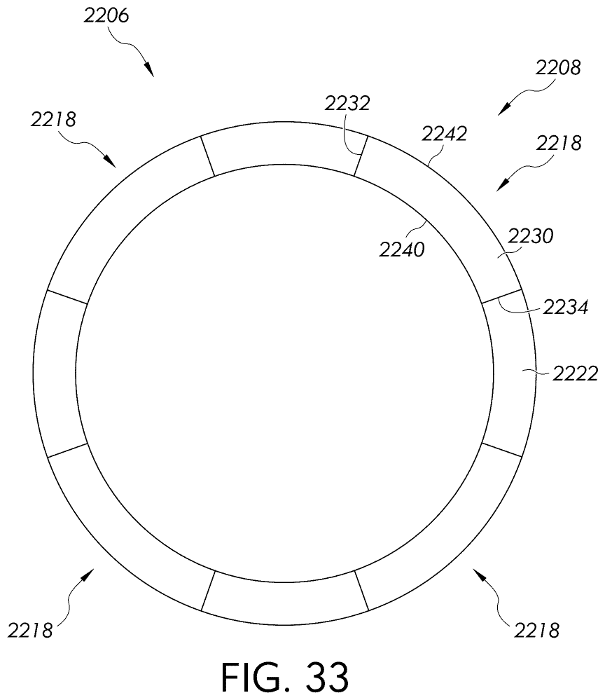

FIGS. 29-33 illustrate a portion of an example bendable catheter body 2200. The example catheter body 2200 is configured to have an enhanced combination of torqueability, flexibility, and resistance to compression and elongation. The catheter body 2200 is bendable or flexible in multiple directions. The catheter body 2200 has a longitudinal axis AL and, in some implementations, has a full 360-degree range of direction of bending relative to the longitudinal axis AL. The catheter body 2200 can be used with any suitable delivery system, such as any delivery system described herein. The catheter body 2200 can be used in a steerable catheter configuration or in a non-steerable catheter configuration.

For example, in some implementations, the catheter body 2200 can be used as part of, or in conjunction with, the steerable catheter 1400 of FIG. 19. The catheter body 2200 can serve as the outer shaft 1402 and/or the inner shaft 1404, can be incorporated as part of the outer shaft 1402 and/or the inner shaft 1404, or can be used in conjunction with the outer shaft 1402 and/or the inner shaft 1404. The catheter body 2200 can be steerable by any suitable steering means, such as any means for steering a steerable catheter disclosed herein. In some implementations, the catheter body 2200 can also be used as part of the exemplary delivery system 2100 of FIGS. 20-28. For example, in some implementations, the catheter body 2200 can be positioned concentrically inside or outside of the plurality of links 2106. The catheter body 2200 can be steerable by any suitable steering means, such as any means for steering a steerable catheter disclosed herein.

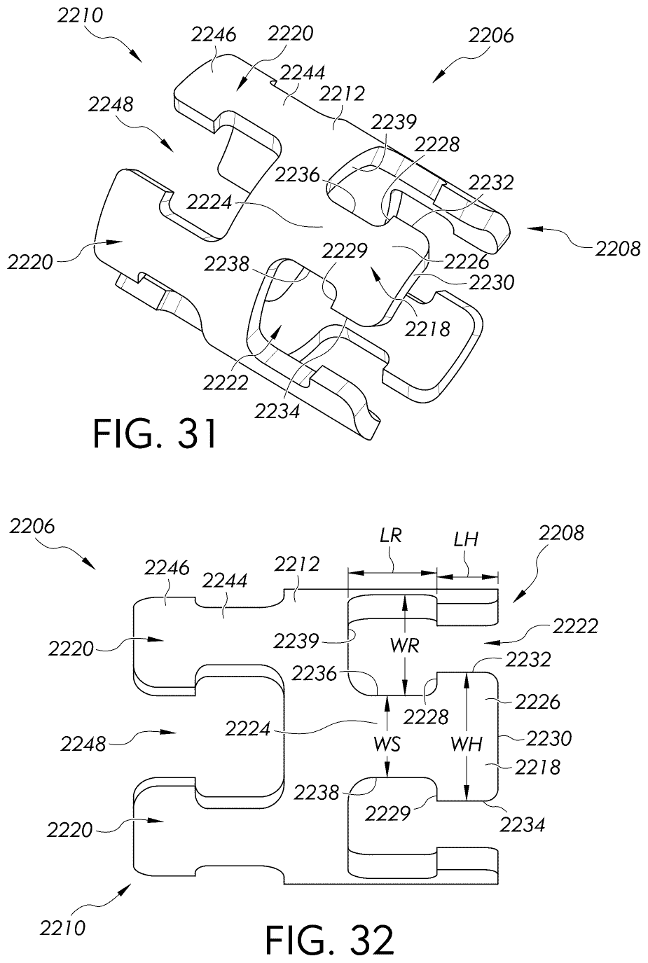

The catheter body 2200 can be configured in a variety of ways. In the illustrated example of FIGS. 29-33, the catheter body 2200 is formed from a plurality of links 2206 that are operatively connected in series along the longitudinal axis AL. The links 2206 are operatively connected to each other such that two operatively connected links can move axially relative to each other and tilt relative to each other while remaining connected. The plurality of links 2206 can be configured in a variety of ways. As shown in FIGS. 31-33, in the illustrated example, each link 2206 includes a proximal end 2208, a distal end 2210 opposite the proximal end 2208, and an intermediate portion 2212 between the proximal end 2208 and the distal end 2210. In some implementations, the intermediate portion 2212 is annular having a circular cross-section. In other implementations, the intermediate portion 2212 can have a cross-section other than circular (e.g., oval, elliptical, etc.).

In some implementations, each link 2206 includes a plurality of first male protrusions 2218 extending proximally from the intermediate portion 2212 and a plurality of second male protrusions 2220 extending distally from the intermediate portion 2212. The first male protrusions 2218 and the second male protrusions 2220 can be configured in variety of ways, including, but not limited to, the shape and size of each protrusion, the number of protrusions, and the arrangement of the protrusions on the intermediate portion and relative to each other. In some implementations, the link 2206 includes four first male protrusions 2218 equally spaced apart around the periphery of the link 2206. In other implementations, however, the link 2206 can have more or less than four first male protrusions 2218. In some implementations, the spaced apart first male protrusions 2218 form four complementary shaped first female recesses 2222 therebetween.

In some implementations, each first male protrusion 2218 has a proximal portion or stem portion 2224 and a distal portion or head portion 2226. The stem portion 2224 has a first width WS and the head portion 2226 has second width WH that is greater than the first width WS. In the illustrated implementations, each first male protrusion 2218 has a T-shape.

The head portion 2226 is connected to the stem portion 2224 via a pair of engagement surfaces (i.e., a first engagement surface 2228 and a second engagement surface 2229) facing the intermediate portion 2212. In some implementations, the first and second engagement surfaces 2228, 2229 extend perpendicular to the longitudinal axis AL. The head portion 2226 includes an end surface 2230 opposite the first and second engagement surfaces 2228, 2229, a first head side surface 2232 extending between the first engagement surface 2228 and the end surface 2230, and a second head side surface 2234 opposite the first head side surface 2232 and extending between the second engagement surface 2229 and the end surface 2230. In some implementations, the first head side surface 2232 is parallel to the second head side surface 2234. In some implementations, the first head side surface 2232 and/or the second head side surface 2234 are perpendicular to the end surface 2230.

In some implementations, the stem portion 2224 includes a first stem side surface 2236 and a second stem side surface 2238 opposite the first stem side surface 2236 and connected by an edge surface 2239 of the intermediate portion 2212. In some implementations, the first stem side surface 2236 is parallel to the second stem side surface 2238. Each of the first male protrusions 2218 includes an inner face 2240 and an outer face 2242. In some implementations that inner face 2240 and the outer face 2242 are parallel. In some implementations, the inner face 2240 is concave and the outer face 2242 is convex.

Each stem portion 2224 has a width WS and each first female recess 2222 has a width WR in the portion of the first female recess 2222 circumferentially adjacent the stem portion 2224. In some implementations, the width WR of the first female recess 2222 is greater than the width WS of the stem portion 2224. Each head portion 2226 has a length LH and each first female recess 2222 has a length LR between the intermediate portion 2212 and the engagement surfaces 2228, 2229. In some implementations, the length LH of the head portion 2226 is less than the length LR of the first female recess 2222. In some implementations, the length LH is 90% of the length LR, is 80% of the length LR, is 75% the length LR, or is 70% the length LR.

In some implementations, the link 2206 includes four second male protrusions 2220 equally spaced apart around the periphery of the link 2206. In other implementations, however, the link 2206 can have more or less than four second male protrusions 2220. In the illustrated examples, the second male protrusions 2220 are configured substantially the same to the first male protrusions 2218, thus the description of the first male protrusions 2218 applies equally to the second male protrusions 2220. For example, each second male protrusions 2220 has a proximal portion or stem portion 2244 and a distal portion or head portion 2246.

In the illustrated implementation, the spaced apart second male protrusions 2220 form four complementary shaped second female recesses 2248 therebetween. In the illustrated example, the second male protrusions 2220 are offset around the periphery of the link 2206 from the first male protrusions 2218. In some implementations, the second male protrusions 2220 are offset to axially align with the first female recesses 2222. In some implementations, therefore, the second male protrusions 2220 are offset 45 degrees from the first male protrusions 2218.

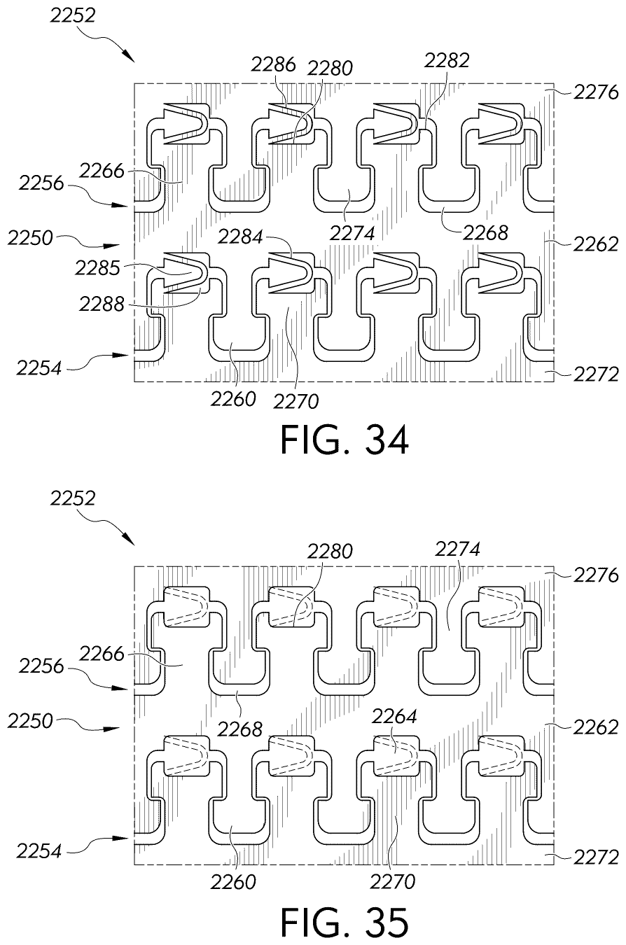

The catheter body 2200 can be configured in a variety of ways. In some implementations, the catheter body 2200 if formed by cutting (e.g., laser cutting) a hypotube. FIGS. 34-36 illustrate a portion of a portion of an example first link 2250 for a catheter body being cut (e.g., laser cut) from a hypotube 2252. The illustrated portion of the hypotube 2252 is shown in plan view for ease of illustration.

Referring to FIG. 34, the hypotube 2252 is shown with a first series of a recurring cut pattern 2254 and a second series of the recurring cut pattern 2256 spaced apart and generally parallel to the first series of the recurring cut pattern 2254. The cut patterns 2254, 2256 can be configured in a variety of ways. In some implementations, the first series of a recurring cut pattern 2254 is configured to form, for the first link 2250, a plurality of first male protrusions 2260 (similar to the first male protrusions 2218 of FIGS. 31-32) extending proximal from an intermediate portion 2262 and a plurality of first female recesses 2264 therebetween (FIG. 35) (similar to the first female recesses 2222 of FIGS. 31-32).

Likewise, the second series of the recurring cut pattern 2256 is configured to form, for the first link 2250, a plurality of second male protrusions 2266 (similar to the second male protrusions 2220 of FIGS. 31-32) extending distally from the intermediate portion 2262 and a plurality of second female recesses 2268 therebetween (similar to the second female recesses 2248 of FIGS. 31-32).

In some implementations, simultaneously, the first series of the recurring cut pattern 2254 forms a plurality of second male protrusions 2270 in a second link 2272 adjacent and operatively connected to the first link 2250. Likewise, the second series of the recurring cut pattern 2256 forms a plurality of first male protrusions 2274 in a third link 2276 adjacent and operatively connected to the first link 2250 and opposite the second link 2272.

In the illustrated example, the second male protrusions 2266 differ from the second male protrusions 2220 of FIGS. 31-32 in that the second male protrusions 2266 have a width WH2 that is greater than the width WH of the second male protrusions 2220. In addition, the second male protrusions 2266 have a recessed portion 2280 in an end surface 2282 of the second male protrusions 2266.

As shown in FIG. 34, the first series of cut patterns 2254 is configured to form a plurality of first bridges 2284. The plurality of first bridges 2284 are configured to keep the first link 2250 and the second link 2272 in place and stable relative to each other during the cutting process. In the illustrated implementation, each of the plurality of first bridges 2284 is formed in the area associated with a corresponding first female recess 2264. In other implementations, however, the plurality of first bridges 2284 can be formed at any location between the first and the second links 2250, 2272.

The plurality of first bridges 2284 can be configured in a variety of ways. In some implementations, each of the plurality of first bridges 2284 is a portion of the hypotube 2252 connecting the first link 2250 and the second link 2272 that is not initially removed during the step of cutting the recuring patterns in the hypotube 2252. In the illustrated implementation, each of the plurality of first bridges 2284 is a V-shaped strip of material extending between and connecting the first link 2250 and the second link 2272.

As shown in FIG. 34, in some implementations, to form each first bridge 2284, each of the first cut patterns 2254 includes a male end 2285 and a complementary female end 2288. The bridge is formed between the male end 2285 of one cut pattern 2254 and the female end 2288 of an adjacent cut pattern 2254. As shown in FIG. 34, a plurality of second bridges 2286, like the first bridges 2284, is formed between the first link 2250 and the third link 2276. Similar bridges can be formed during each pair of adjacent links during manufacture.

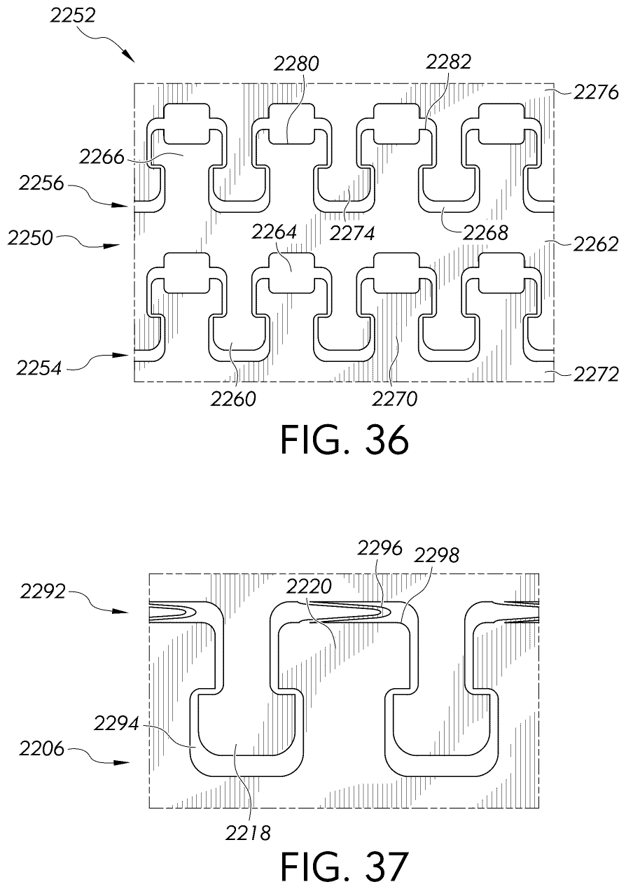

Referring to FIGS. 35-36, once the plurality of first cut patterns 2254, the plurality of second cut patterns 2256, and any additional series of cut patterns have been cut into the hypotube 2252, the plurality of first bridges 2284, the plurality of second bridges 2286, and any additional bridges are cut from the hypotube 2252, as shown by the dashed lines in FIG. 35.

FIG. 37 illustrates a partial view of a hypotube 2292 in a first stage of manufacturing the link 2206 of FIGS. 31-32. The link 2206 can be manufactured by cutting recurring cut patterns in the hypotube 2292 as described regarding the link 2250. For example, a recurring cut pattern 2294 can be used to form the second male protrusions 2220 and a plurality of first bridges 2296 similar to the first bridges 2284 of FIGS. 34-36. The recurring cut pattern 2294, however, differs from the recurring cut pattern 2256 of FIGS. 34-36. In particular, the recurring cut pattern 2294 forms a thinner plurality of first bridges 2296 that eliminate, or minimize, a recess (e.g., 2280 of FIG. 36) in an end surface 2298 of the second male protrusions 2220. Thus, when the plurality of first bridges 2296 are removed, the second male protrusions 2220 can be substantially similar to the first male protrusions 2218.

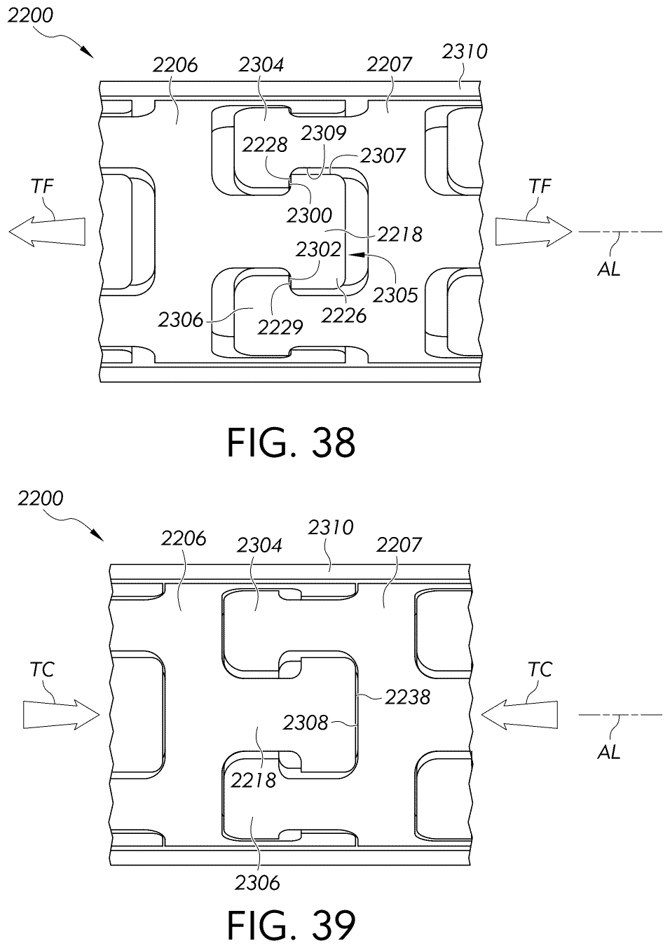

FIGS. 38-39 illustrate a portion of the catheter body 2200 with the first link 2206 and a second link 2207 in an expanded state and in a compressed state, respectively. Referring to FIG. 38, when the catheter body 2200 is exposed to an adequate axial tensile force TF, the tensile force TF pulls the first link 2206 and the second link 2207 away from each other until the pair of engagement surfaces (i.e., the first engagement surface 2228 and the second engagement surface 2229) on each of the first male protrusions 2218 engage a similar engagement surfaces (i.e., a first engagement surface 2300 and a second engagement surface 2302) on two second male protrusions 2304, 2306 of the second link 2207. In the illustrated implementation, the engagement surfaces 2228, 2229, 2300, 2302 extend perpendicular to the longitudinal axis AL of the catheter body 2200. As a result, once the engagement surfaces of the first link 2206 engage corresponding engagement surfaces of the second link 2207, and so on, the catheter body 2200 provides a high resistance to further elongation.

Further, the operatively connected first link 2206 and second link 2207 provide high torqueability. For example, the head portion 2226 of each of the first male protrusion 2218 is received within a corresponding female recess 2305 of the second link 2207. When torque is applied to the catheter body 2200, a side surface 2307 of the head portion 2226 (e.g., the first head side surface 2232) engages a side surface 2309 of the corresponding female recess 2305 (e.g., similar to the first stem side surface 2236 of the first link 2206) thereby transferring the torque along the catheter body 2200.

Referring to FIG. 39, when the catheter body 2200 is exposed to adequate axial compression forces TC, the compression forces TC push the first link 2206 and the second link 2207 toward each other until the end surface 2230 each of the first male protrusions 2218 engage an edge surface 2308 on the second link 2207 (similar to the edge surface 2239 of the first female recesses 2222 of FIG. 31). As a result, once the end surfaces of the male protrusions 2218, 2220 of the first link 2206 engage corresponding edge surfaces on adjacent links, and so on, the catheter body 2200 provides a high resistance to further compression.

In some implementations, an outer layer 2310 can be used in combination with the catheter body 2200. The outer layer 2310 can be configured to preset the catheter body 2200 to have distance limits in extension and compression (i.e., limit the axial movement between the plurality of links of the catheter body 2200). The outer layer 2310 can be configured in a variety of ways. In some implementations, the outer layer 2310 is a polymer lamination.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

While various inventive aspects, concepts and features of the disclosures may be described and illustrated herein as embodied in combination in the example implementations, these various aspects, concepts, and features may be used in many alternative implementations, either individually or in various combinations and sub-combinations thereof. Unless expressly excluded herein all such combinations and sub-combinations are intended to be within the scope of the present application. Still further, while various alternative implementations as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative implementations, whether presently known or later developed. Those skilled in the art may readily adopt one or more of the inventive aspects, concepts, or features into additional implementations and uses within the scope of the present application even if such implementations are not expressly disclosed herein.

Additionally, even though some features, concepts, or aspects of the disclosures may be described herein as being a preferred arrangement or method, such description is not intended to suggest that such feature is required or necessary unless expressly so stated. Still further, exemplary or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of exemplary methods or processes are not limited to inclusion of all steps as being required in all cases, nor is the order that the steps are presented to be construed as required or necessary unless expressly so stated. The words used in the claims have their full ordinary meanings and are not limited in any way by the description of the implementations in the specification.

### Examples

Example 1. A catheter body, comprising:
a plurality of links connected in series along a longitudinal axis, wherein the plurality of links include a first link comprising:
   an annular first intermediate portion;
   a first male protrusion extending proximally from the first intermediate portion;
   a second male protrusion extending distally from the first intermediate portion;
   wherein the first male protrusion includes a pair of first engagement surfaces facing the first intermediate portion and a first end surface facing opposite the pair of first engagement surfaces;
wherein the first male protrusion is configured to be received within a first female recess of a second link of the plurality of links;
wherein the pair of first engagement surfaces are configured to engage a pair of second engagement surfaces of the second link to limit axial extension of the catheter body; and
wherein the first end surface is configured to engage an edge surface of a second intermediate portion of the second link to limit axial compression of the catheter body.

Example 2. The catheter body of claim 1, wherein the pair of first engagement surfaces are perpendicular to the longitudinal axis.

Example 3. The catheter body of claim 1 or 2, wherein the first male protrusion has a head portion and a stem portion positioned between the head portion and the first intermediate portion, wherein the head portion has a first width and the stem portion has a second width less than the first width.

Example 4. The catheter body of claim 3, wherein the head portion includes a first head side surface and a second head side surface opposite of and parallel to the first head side surface.

Example 5. The catheter body of claim 3 or 4, wherein the pair of first engagement surfaces connects the stem portion to the head portion.

Example 6. The catheter body of any of claims 3-5, wherein the stem portion includes a first stem side surface and a second stem side surface opposite of and parallel to the first stem side surface.

Example 7. The catheter body of any of claims 1-6, wherein the first male protrusion is one of a plurality of first male protrusions evenly spaced apart around a periphery of the first intermediate portion.

Example 8. The catheter body of claim 7, wherein the plurality of first male protrusions includes four first male protrusions.

Example 9. The catheter body of claim 8, wherein the second male protrusion is one of a plurality of second male protrusions evenly spaced apart around the periphery of the first intermediate portion.

Example 10. The catheter body of claim 9, wherein the plurality of second male protrusions are circumferentially offset from the plurality of first male protrusions.

Example 11. The catheter body of claim 10, wherein the plurality of second male protrusions are circumferentially offset from the plurality of first male protrusions by 45 degrees.

Example 12. The catheter body of any of claims 1-11, wherein the first link is axially moveable relative to the second link.

Example 13. The catheter body of any of claims 1-12, wherein the first link is tiltable relative to the second link.

Example 14. The catheter body of any of claims 1-13, wherein the first male protrusion is T-shaped.

Example 15. The catheter body of any of claims 1-14, wherein the second link comprises:
a third male protrusion extending distally from the second intermediate portion;
a fourth male protrusion extending distally from the second intermediate portion; and
wherein the second intermediate portion, the third male protrusion, and the fourth male protrusion define the first female recess of the second link.

Example 16. The catheter body of claim 15, wherein the pair of second engagement surfaces of the second link include one of the pair of second engagement surfaces on the third male protrusion and a second of the pair of second engagement surfaces on the fourth male protrusion.

Example 17. The catheter body of claim 16, wherein the pair of second engagement surfaces are parallel to each other.

Example 18. A delivery system for delivering a medical device to a desired location, the delivery system comprising:
a catheter having a bendable catheter body;
at least one actuation element, a distal end of said at least one actuation element connected at a distal end to the bendable catheter body; and
a handle comprising a steering mechanism, said handle attached to the catheter at a proximal end of the catheter, a proximal end of the at least one actuation element connected to the steering mechanism, wherein the steering mechanism is capable of applying a force to the at least one actuation element to bend the catheter body;
wherein the catheter body comprises:
a plurality of links connected in series along a longitudinal axis, wherein the plurality of links include a first link comprising:
   an annular first intermediate portion;
   a first male protrusion extending proximally from the first intermediate portion;
   a second male protrusion extending distally from the first intermediate portion;
   wherein the first male protrusion includes a pair of first engagement surfaces facing the first intermediate portion and a first end surface facing opposite the pair of first engagement surfaces;
wherein the first male protrusion is configured to be received within a first female recess of a second link of the plurality of links;
wherein the pair of first engagement surfaces are configured to engage a pair of second engagement surfaces of the second link to limit axial extension of the catheter body; and
wherein the first end surface is configured to engage an edge surface of a second intermediate portion of the second link to limit axial compression of the catheter body.

Example 19. The delivery system of claim 18, wherein the pair of first engagement surfaces are perpendicular to the longitudinal axis.

Example 20. The delivery system of claim 18 or 19, wherein the first male protrusion has a head portion and a stem portion positioned between the head portion and the first intermediate portion, wherein the head portion has a first width and the stem portion has a second width less than the first width.

Example 21. The delivery system of claim 20, wherein the head portion includes a first head side surface and a second head side surface opposite of and parallel to the first head side surface.

Example 22. The delivery system of claim 20 or 21, wherein the pair of first engagement surfaces connects the stem portion to the head portion.

Example 23. The delivery system of any of claims 20-22, wherein the stem portion includes a first stem side surface and a second stem side surface opposite of and parallel to the first stem side surface.

Example 24. The delivery system of claims 18-23, wherein the first male protrusion is one of a plurality of first male protrusions evenly spaced apart around a periphery of the first intermediate portion.

Example 25. The delivery system of claim 24, wherein the plurality of first male protrusions includes four first male protrusions.

Example 26. The delivery system of claim 25, wherein the second male protrusion is one of a plurality of second male protrusions evenly spaced apart around the periphery of the first intermediate portion.

Example 27. The delivery system of claim 26, wherein the plurality of second male protrusions are circumferentially offset from the plurality of first male protrusions.

Example 28. The delivery system of claim 27, wherein the plurality of second male protrusions are circumferentially offset from the plurality of first male protrusions by 45 degrees.

Example 29. The delivery system of any of claims 18-28, wherein the first link is axially moveable relative to the second link.

Example 30. The delivery system of any of claims 18-29, wherein the first link is tiltable relative to the second link.

Example 31. The delivery system of any of claims 18-30, wherein the first male protrusion is T-shaped.

Example 32. The delivery system of any of claims 18-31, wherein the second link comprises:
a third male protrusion extending distally from the second intermediate portion;
a fourth male protrusion extending distally from the second intermediate portion; and
wherein the second intermediate portion, the third male protrusion, and the fourth male protrusion define the first female recess of the second link.

Example 33. The delivery system of claim 32, wherein the pair of second engagement surfaces of the second link include one of the pair of second engagement surfaces on the third male protrusion and a second of the pair of second engagement surfaces on the fourth male protrusion.

Example 34. The delivery system of claim 33, wherein the pair of second engagement surfaces are parallel to each other.

Example 35. The delivery system of any of claims 18-34, wherein the at least one actuation element is a pull wire.

What is claimed is:

1. A catheter body, comprising:
a plurality of links connected in series along a longitudinal axis, wherein the plurality of links include a first link comprising:
an annular first intermediate portion;
a first male protrusion extending proximally from the first intermediate portion;
a second male protrusion extending distally from the first intermediate portion;
wherein the first male protrusion includes a pair of first engagement surfaces facing the first intermediate portion and a first end surface facing opposite the pair of first engagement surfaces;
wherein the first male protrusion is configured to be received within a first female recess of a second link of the plurality of links;
wherein the pair of first engagement surfaces are configured to engage a pair of second engagement surfaces of the second link to limit axial extension of the catheter body; and
wherein the first end surface is configured to engage an edge surface of a second intermediate portion of the second link to limit axial compression of the catheter body.

2. The catheter body of claim 1, wherein the pair of first engagement surfaces are perpendicular to the longitudinal axis.

3. The catheter body of claim 1, wherein the first male protrusion has a head portion and a stem portion positioned between the head portion and the first intermediate portion, wherein the head portion has a first width and the stem portion has a second width less than the first width.

4. The catheter body of claim 3, wherein the head portion includes a first head side surface and a second head side surface opposite of and parallel to the first head side surface.

5. The catheter body of claim 3, wherein the pair of first engagement surfaces connects the stem portion to the head portion.

6. The catheter body of claim 3, wherein the stem portion includes a first stem side surface and a second stem side surface opposite of and parallel to the first stem side surface.

7. The catheter body of claim 1, wherein the first male protrusion is one of a plurality of first male protrusions evenly spaced apart around a periphery of the first intermediate portion.

8. The catheter body of claim 7, wherein the plurality of first male protrusions includes four first male protrusions.

9. The catheter body of claim 8, wherein the second male protrusion is one of a plurality of second male protrusions evenly spaced apart around the periphery of the first intermediate portion.

10. The catheter body of claim 9, wherein the plurality of second male protrusions are circumferentially offset from the plurality of first male protrusions.

11. The catheter body of claim 10, wherein the plurality of second male protrusions are circumferentially offset from the plurality of first male protrusions by 45 degrees.

12. The catheter body of claim 1, wherein the second link comprises:
a third male protrusion extending distally from the second intermediate portion;
a fourth male protrusion extending distally from the second intermediate portion; and
wherein the second intermediate portion, the third male protrusion, and the fourth male protrusion define the first female recess of the second link.

13. The catheter body of claim 12, wherein the pair of second engagement surfaces of the second link include one of the pair of second engagement surfaces on the third male protrusion and a second of the pair of second engagement surfaces on the fourth male protrusion.

14. The catheter body of claim 13, wherein the pair of second engagement surfaces are parallel to each other.

15. A delivery system for delivering a medical device to a desired location, the delivery system comprising:
a catheter having a bendable catheter body;
at least one actuation element, a distal end of said at least one actuation element connected at a distal end to the bendable catheter body; and
a handle comprising a steering mechanism, said handle attached to the catheter at a proximal end of the catheter, a proximal end of the at least one actuation element connected to the steering mechanism, wherein the steering mechanism is capable of applying a force to the at least one actuation element to bend the catheter body;
wherein the catheter body comprises:
a plurality of links connected in series along a longitudinal axis, wherein the plurality of links include a first link comprising:
an annular first intermediate portion;
a first male protrusion extending proximally from the first intermediate portion;
a second male protrusion extending distally from the first intermediate portion;
wherein the first male protrusion includes a pair of first engagement surfaces facing the first intermediate portion and a first end surface facing opposite the pair of first engagement surfaces;
wherein the first male protrusion is configured to be received within a first female recess of a second link of the plurality of links;
wherein the pair of first engagement surfaces are configured to engage a pair of second engagement surfaces of the second link to limit axial extension of the catheter body; and
wherein the first end surface is configured to engage an edge surface of a second intermediate portion of the second link to limit axial compression of the catheter body.

16. The delivery system of claim 15, wherein the first male protrusion has a head portion and a stem portion positioned between the head portion and the first intermediate portion, wherein the head portion has a first width and the stem portion has a second width less than the first width.

17. The delivery system of 15, wherein the first male protrusion is one of a plurality of first male protrusions evenly spaced apart around a periphery of the first intermediate portion.

18. The delivery system of claim 17, wherein the plurality of first male protrusions includes four first male protrusions.

19. The delivery system of claim 17, wherein the second male protrusion is one of a plurality of second male protrusions evenly spaced apart around the periphery of the first intermediate portion.

20. The delivery system of claim 19, wherein the plurality of second male protrusions are circumferentially offset from the plurality of first male protrusions by 45 degrees.
